(19) United States
(12) Patent Application Publication
(10) Pub. No.: US 2018/0125597 A1
(43) Pub. Date: May 10, 2018

(54) STERILE BOUNDARY BETWEEN A ROBOT AND A SURGICAL FIELD

(71) Applicants: Emily Gogarty, Montreal (CA); Benoit Pelletier, Laval (CA); Pierre Couture, Montreal (CA)

(72) Inventors: Emily Gogarty, Montreal (CA); Benoit Pelletier, Laval (CA); Pierre Couture, Montreal (CA)

(21) Appl. No.: 15/803,247

(22) Filed: Nov. 3, 2017

Related U.S. Application Data
(60) Provisional application No. 62/417,740, filed on Nov. 4, 2016.

(51) Int. Cl. A61B 46/10; A61B 34/30; A61B 90/00

(52) U.S. Cl. CPC A61B 46/10; A61B 90/05; A61B 34/30

(57) ABSTRACT

Embodiments of a system and method for preparing a robot for use within a sterile surgical environment are generally described herein. A system may include a sterile robotic drape having a first sterile side and a second non-sterile side opposite the first sterile side, a robot interface embedded in the sterile robotic drape, the robot interface including a first face on the first sterile side and a second face on the second non-sterile side, and a plurality of anchors on the second face of the robot interface.

### CLAIM OF PRIORITY

This application claims the benefit of U.S. Provisional Patent Application Ser. No. 62/417,740, filed on Nov. 4, 2016, the benefit of priority of which is claimed hereby, and which is incorporated by reference herein in its entirety.

### BACKGROUND

From fully autonomous robots to surgeon-controlled robots, the use of robotics in surgery is on the rise. As these uses become more complicated and intricate, techniques are becoming dependent on these robotics to ensure successful surgeries. To use these robotics in multiple different surgeries or for multiple different patients, sterilizing the robotics can be cumbersome, time consuming, or impossible. Disposable robotics parts may be cost prohibitive or made of inferior parts, increasing the likelihood of complications in a surgery.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1B illustrate a system for preparing a robot for use within a sterile surgical environment in accordance with some embodiments.

FIG. 2 illustrates a cross-sectional view of coupling components of a robotic arm and an embedded robot interface in accordance with some embodiments.

FIG. 3 illustrates a cross-sectional view of coupling components of a robotic arm, an anchor (e.g., of an embedded robot interface), and a screw in accordance with some embodiments.

FIG. 4 illustrates a robotic system for use within a sterile surgical environment in accordance with some embodiments.

FIG. 5 illustrates a flow chart showing a technique for preparing a robot for use within a sterile surgical environment in accordance with some embodiments.

FIGS. 6A-6B illustrate sterile draping components in accordance with some embodiments.

FIG. 7 illustrates a sterile draping system with a cone in accordance with some embodiments.

FIG. 8 illustrates a sterile draping system with a ring in accordance with some embodiments.

FIGS. 9A-9B illustrate a sterile drape and plate system in accordance with some embodiments.

### DETAILED DESCRIPTION

A sterile surgical field is an essential part of modern surgery. As the use of robotics in surgery increases, maintaining that sterile surgical field becomes more and more difficult. Parts of a robot system may be difficult or impossible to sterilize. To maintain a sterile surgical field, the systems and methods described herein make use of a sterile robotic drape. The sterile robotic drape may have a first sterile side and a second non-sterile side opposite the first sterile side, to separate a sterile surgical field from non-sterile robotics. For example, a non-sterile robotic arm may be separated from a sterile robotic end effector. The non-sterile robotic arm may be used to control movement of the sterile robotic end effector without compromising the sterility of the surgical field. The sterile robotic end effector may be used in surgery to aid a surgeon, complete a task, perform a technique, or the like.

The sterile robotic drape may act as a barrier between the non-sterile robotic arm and the sterile robotic end effector. For example, the sterile robotic drape may cover a portion of the robotic arm (e.g., drape over the portion). In an example, the sterile robotic drape may be secured in the surgical field to act as a barrier. The sterile robotic drape may be made of plastic, may be elastic, or may be configured to stick to the robotic arm. In an example, the sterile robotic drape may be sterilized before use or may be disposable.

The sterile robotic drape may include an embedded robot interface to couple the robotic arm to the sterile robotic end effector. The embedded robot interface may include a sterile side and a non-sterile side, the sterile side to interface with the sterile robotic end effector and the non-sterile side to interface with a portion of the robotic arm. The embedded robot interface may include a plurality of anchors to couple with a plurality of apertures in the robotic arm, the plurality of anchors to receive a plurality of screws to secure the embedded robot interface to the sterile robotic end effector (e.g., through a plurality of apertures in the sterile robotic end effector). An alignment peg, such as a stand-alone peg or a peg affixed to the sterile robotic end effector, the embedded robot interface, or the robotic arm may be used to align the robotic arm, the embedded robot interface, and the sterile robotic end effector. In an example, two or more alignment pegs may be used, such as to restrict rotation among components.

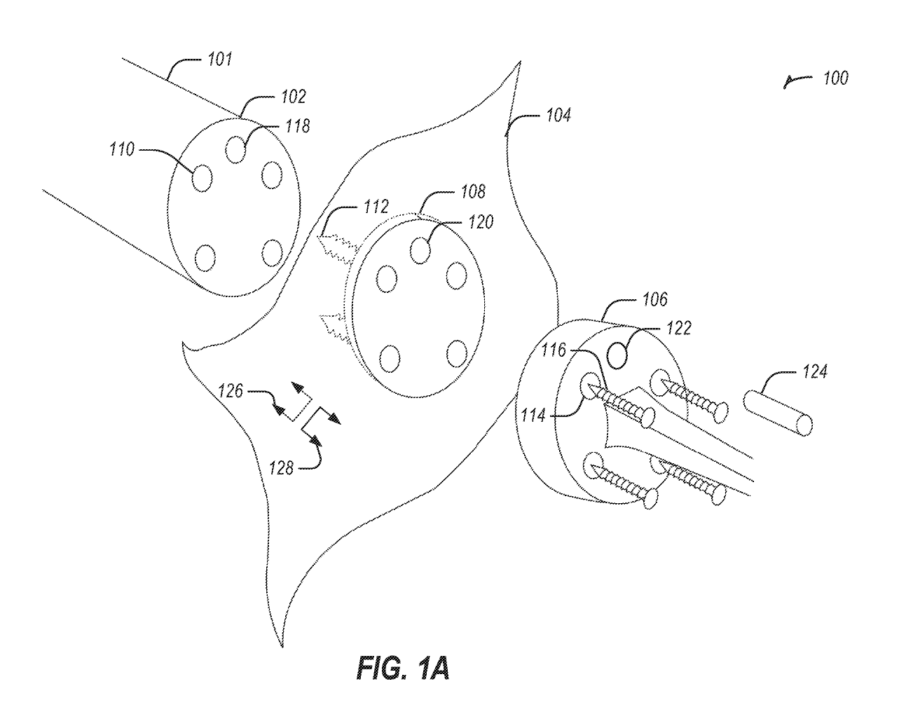

FIGS. 1A-1B illustrate a system 100 for preparing a robot for use within a sterile surgical environment in accordance with some embodiments. FIG. 1A illustrates a projection view of the system 100. FIG. 1B illustrates a cross-section side view of the system 100. The system 100 includes a robotic arm 101, a sterile robotic drape 104 and a sterile robotic end effector 106. The robotic arm 101 includes an end effector receiver 102 including a plurality of apertures (e.g., an aperture 110 or a first alignment aperture 118). The sterile robotic drape 104 includes an embedded robot interface 108, a first sterile side 128, and a second non-sterile side 126 opposite the first sterile side.

The embedded robot interface 108 may include a plurality of anchors (e.g., anchor 112). The plurality of anchors may project from the second non-sterile side 126 of the sterile robotic drape 104. The embedded robot interface 108 may include a second alignment aperture 120. The plurality of anchors may be configured to secure the embedded robot interface 108 to the robotic arm 101 by coupling with the plurality of apertures (e.g., aperture 110) in the end effector receiver 102.

The sterile robotic end effector 106 may include a plurality of apertures (e.g., an aperture 114 or a third alignment aperture 122). In an example, the third alignment aperture 122 may be replaced by an alignment peg integrated into the sterile robotic end effector 106, the alignment peg configured to be received by the end effector receiver 102 via the embedded robot interface 108, such as through the first alignment aperture 118 and the second alignment aperture 120. The system 100 may include an alignment peg 124, which is configured to be received by the end effector receiver 102 via the embedded robot interface 108 and the sterile robotic end effector 106, such as through the first alignment aperture 118, the second alignment aperture 120, and the third alignment aperture 122. While a single alignment peg 124 is shown, two or more alignment pegs may be used, such as to restrict rotation among the sterile robotic end effector 106, the embedded robot interface 108, the end effector receiver 102, or the robotic arm 101.

The system 100 may include a plurality of screws (e.g., screw 116). The plurality of screws may secure the sterile robotic end effector 106 to the embedded robot interface 108 by screwing into the plurality of anchors (e.g. anchor 112). In an example, the plurality of screws may cause the plurality of anchors (e.g. anchor 112) to expand, thereby securing the plurality of anchors (e.g. anchor 112) into the plurality of apertures (e.g., aperture 110) of the end effector receiver 102. By causing the plurality of anchors (e.g. anchor 112) to expand and secure into the plurality of apertures (e.g. aperture 110), the plurality of screws may cause the embedded robot interface 108 to securely couple with the robotic arm 101, thereby securing the sterile robotic end effector 106 to the robotic arm 101 via the embedded robot interface 108 (e.g., with the sterile robotic drape 104 separating the robotic arm 101 from the sterile robotic end effector 106). In an example, the plurality of screws or the alignment peg 124 may be used to prevent rotation of one of the robotic arm 101, the embedded robot interface 108, or the sterile robotic end effector 106 from rotating relative to another one or the other two. In an example, two or more components (e.g., 101, 104, 106, or 108) may rotate as a system when connected. In another example, the sterile robotic drape 104 may be configured such that it does not rotate with respect to the embedded robot interface 108. In this example, the robotic arm 101, the embedded robot interface 108, or the sterile robotic end effector 106 may rotate together, and may rotate with respect to the sterile robotic drape 104, which may be held steady. In an example, gaps may be sealed (e.g., using sealant, glue, etc.). The sealed gaps may include areas around the plurality screws or the alignment peg 124.

In an example, the sterile robotic drape 104 may be draped around the robotic arm 101, such as around an arm portion extending away from a distal end of the robotic arm, the distal end including the plurality of apertures (e.g., aperture 110). In an example, the sterile robotic drape 104 may be secured around the robotic arm 101 a distance away from the distal end. In another example, the sterile robotic drape 104 may loosely drape around the robotic arm 101.

The sterile robotic end effector 106 may be configured to couple to the end effector receiver of the robotic arm 101 via the embedded robot interface 108. The robotic arm 101 may be non-sterile and configured to connect (via the end effector receiver 102) to the embedded robot interface 108 on the second non-sterile side 126. The sterile robotic end effector 106 may be configured to couple to the embedded robot interface 108 on the first sterile side 128 of the sterile robotic drape 104. In an example, the sterile robotic end effector 106 includes a cup configured to fit around the embedded robot interface 108.

The robotic arm 101, the sterile robotic drape 104, and the sterile robotic end effector 106 may be separate components configured to be used with different systems. For example, the sterile robotic drape 104 may be configured to be inserted into a separately manufactured robotic system. In another example, a plurality of sterile robotic end effectors may be interchangeably used with the sterile robotic drape 104 and the robotic arm 101. For example, different sterile robotic end effectors may be used for respective surgical procedures. The different sterile robotic end effectors may be changed on the fly, such as without disconnecting the end effector receiver 102 from the sterile robotic drape 104. In an example, the sterile robotic drape 104 may be disposable, such as a drape to be used for a specific patient and then discarded.

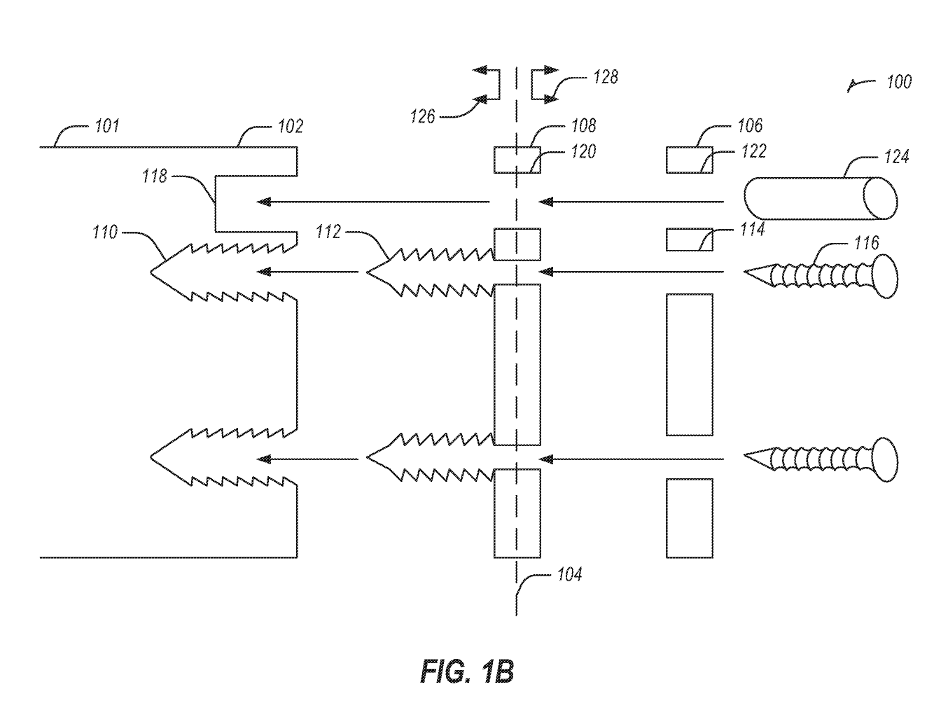

FIG. 2 illustrates a cross-sectional view 200 of coupling components of a robotic arm 206 and an embedded robot interface 212 in accordance with some embodiments. The embedded robot interface 212 includes an anchor 202, and may include a plurality of anchors. The robotic arm 206 includes an aperture 204 configured to receive the anchor 202 to secure the embedded robot interface 212 to the robotic arm 206. For example, the anchor 202 may be inserted into the aperture 204 and prevented from being removed from the aperture 204 unless a particular force is applied to the anchor 202.

In an example, the robotic arm 206 includes a first alignment aperture 210 and the embedded robot interface 212 includes a second alignment aperture 214. An alignment peg 208 may be used to align the embedded robot interface 212 with the robotic arm 206 using the first alignment aperture 210 and the second alignment aperture 215.

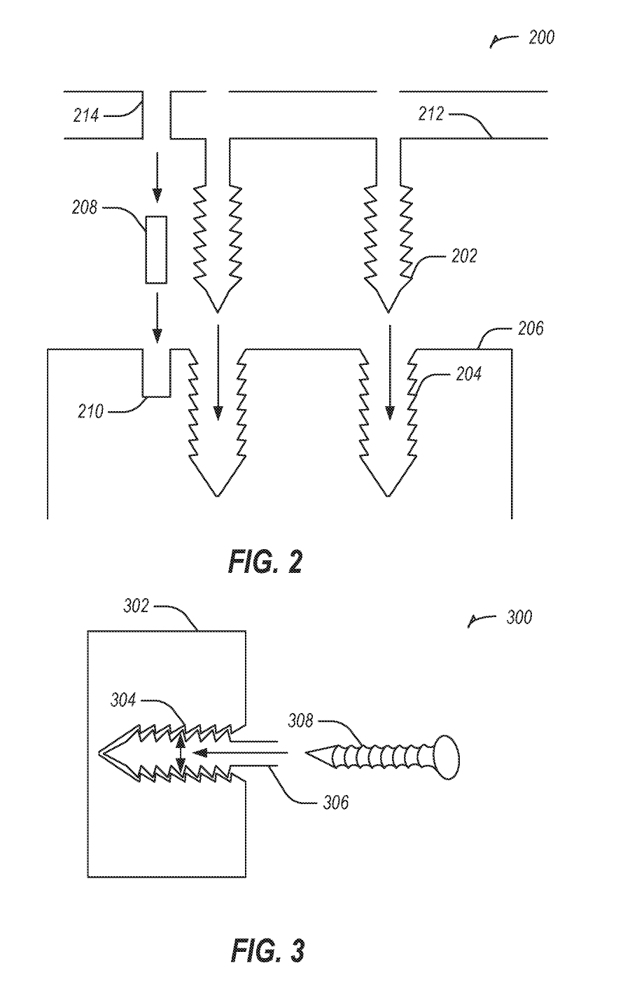

FIG. 3 illustrates a cross-sectional view 300 of coupling components of a robotic arm 302, an anchor 306 (e.g., of an embedded robot interface), and a screw 308 in accordance with some embodiments. The robotic arm 302 may include an aperture 304 or anchor configured to receive the anchor 306 (e.g., to secure an embedded robot interface to the robotic arm 302). The screw 308 may be used to secure a component (e.g., a sterile robotic end effector—not shown) to the robotic arm 302 (e.g., via an embedded robot interface). For example, the screw 308 may be screwed into the anchor 306. The screw 308, may cause the anchor 306 to expand within the aperture 304. The expanded anchor 306 may securely couple with the aperture 304 and the screw 308, such that the robotic arm 302 is coupled to the component (e.g., a sterile robotic end effector) via an embedded robot interface including the anchor 306. In an example, the embedded robot interface may be embedded in a sterile robotic drape, which is secured to the robotic arm and the component (e.g., a sterile robotic end effector). In an example, a sterile environment may include the screw 308 and an inside of the aperture 304 of a first sterile side of the embedded robot interface. A non-sterile environment may include the aperture 304 and the robotic arm 302 generally, and may include an outside of the aperture 304.

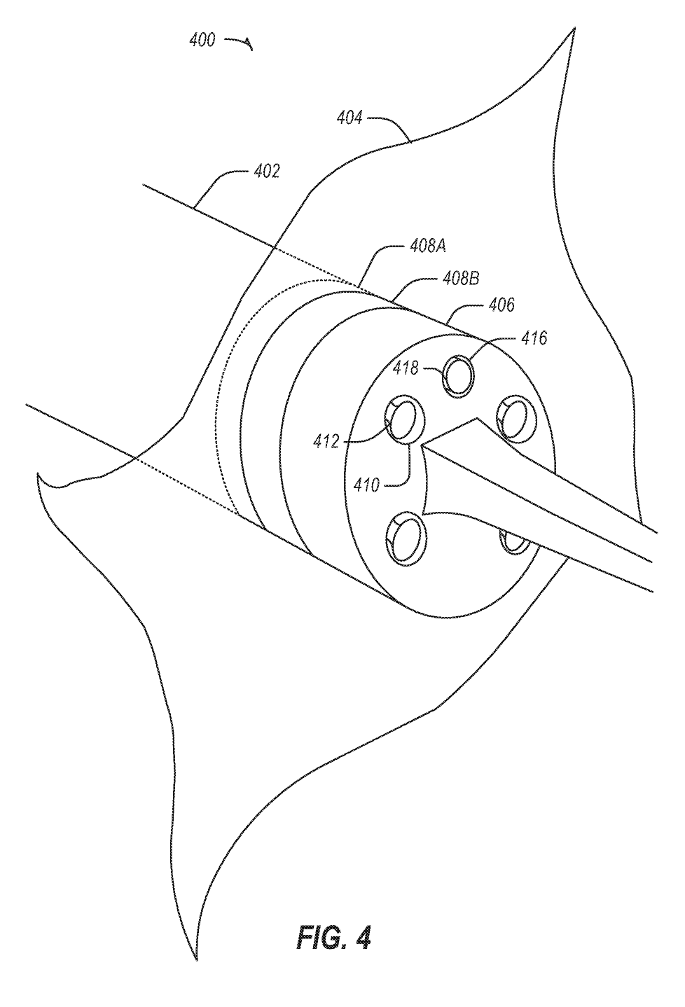

FIG. 4 illustrates a robotic system 400 for use within a sterile surgical environment in accordance with some embodiments. The robotic system 400 includes a robotic arm 402, a sterile robotic drape 404, a non-sterile side of an embedded robot interface 408A, a sterile side of the embedded robot interface 408B, and a sterile robotic end effector 406. The robotic system 400 components may be coupled together, for example using a screw 412 through an aperture-anchor receiver 410. The robotic system 400 may be aligned using an alignment peg 418 and a plurality of alignment apertures 416 (e.g., an alignment aperture for each of the robotic arm 402, the embedded robot interface, and the sterile robotic end effector 406). In an example, the alignment peg 418 may be integrated into the sterile robotic end effector 406, and the plurality of alignment apertures 416 may be integrated into the robotic arm 402 and the embedded robot interface.

The robotic system 400 may include a plurality of sterile components, such as the sterile side of the embedded robot interface 408B, the sterile robotic end effector 406, the plurality of alignment apertures 416, the screw 412, or the aperture-anchor receiver 410. The robotic system 400 may include a plurality of non-sterile components, such as the robotic arm 402 or the non-sterile side of the embedded robot interface 408A. In an example, the components shown in FIG. 4 (e.g., 402, 404, 408A-408B, 406) may rotate as a system when connected. In another example, the sterile robotic drape 404 may be configured such that it does not rotate with respect to the embedded robot interface (e.g., with sterile side 408B and non-sterile side 408A). In this example, the robotic arm 402, the embedded robot interface, and the sterile robotic end effector 406 may rotate together, and may rotate with respect to the sterile robotic drape 404, which may be held steady.

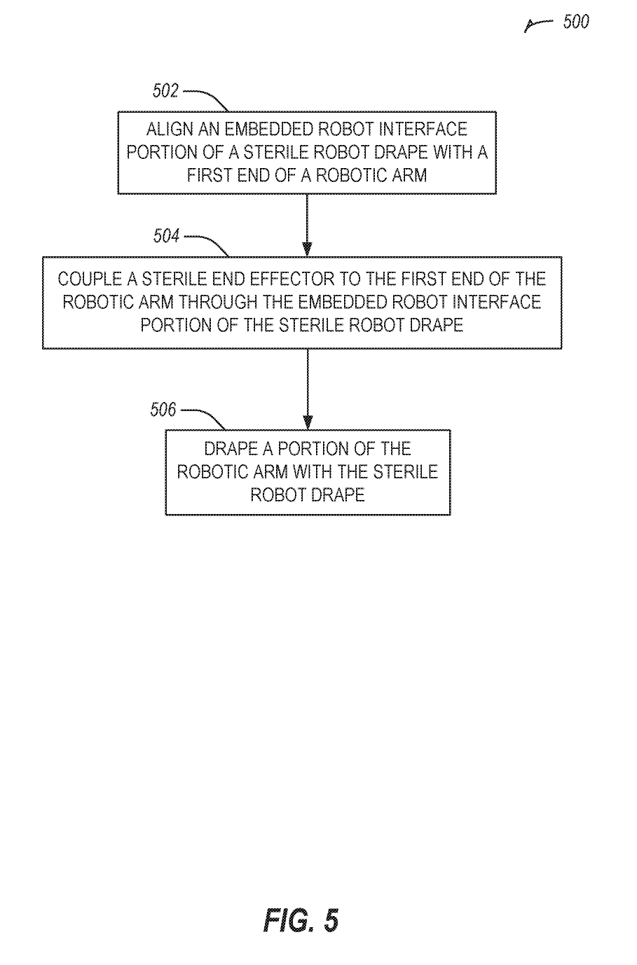

FIG. 5 illustrates a flow chart showing a technique 500 for preparing a robot for use within a sterile surgical environment in accordance with some embodiments. The technique 500 includes an operation 502 to align an embedded robot interface portion of a sterile robot drape with a first end of a robotic arm. The first end of the robotic arm may include an end effector receiver to receive anchors of the embedded robot interface portion of the sterile robot drape.

The technique 500 includes an operation 504 to couple a sterile end effector to the first end of the robotic arm through the embedded robot interface portion of the sterile robot drape. Operation 504 may thereby secure the sterile robot drape to the robotic arm. For example, the embedded robot interface portion may include a plurality of anchors that may couple with a plurality of apertures on the first end of the robotic arm. In an example, the sterile end effector may include a plurality of apertures to receive a plurality of screws. The plurality of screws may be used in the technique 500 to attach the sterile end effector to the embedded robot interface portion by screwing the screws into the anchors. The screws may cause the anchors to expand in the plurality of apertures in the first end of the robotic arm as the screws are tightened, thereby securing the sterile end effector to the robotic arm.

The technique 500 includes an operation 506 to drape a portion of the robotic arm with the sterile robot drape, such as a portion of the robotic arm extending from the first end of the robotic arm. The sterile robot drape may be configured or cut to conform to dimensions of the robotic arm such that a portion of the robotic arm is covered by the sterile robot drape in a surgical field. The portion of the robotic arm that is covered may include any portion of the robotic arm that is in the surgical field.

In an example, the technique 500 includes coupling the sterile end effector to the first end of the robotic arm through the embedded robot interface portion of the sterile robot drape by coupling the robotic arm to a non-sterile side of the embedded robot interface portion and coupling the sterile end effector to a sterile side of the embedded robot interface portion. The non-sterile side of the embedded robot interface portion may correspond with a non-sterile side of the sterile robot drape, and the sterile side of the embedded robot interface portion may correspond with a sterile side of the sterile robot drape. The non-sterile side of the embedded robot interface portion may include a plurality of anchors that may be coupled with a plurality of anchors on the first end of the robotic arm. The sterile side of the embedded robot interface portion may include a plurality of openings corresponding to the plurality of anchors, the plurality of openings to receive a plurality of screws. In an example, the technique 500 includes coupling the sterile end effector to the first end of the robotic arm through the embedded robot interface portion of the sterile robot drape by securing a plurality of anchors on the embedded robot interface portion to a plurality of apertures in the first end of the robotic arm using a plurality of screws. The plurality of screws may be sterile, and the technique 500 may include screwing the plurality of screws into the plurality of anchors through the plurality of openings to connect the sterile side of the embedded robot interface portion to the sterile end effector. Respective insides of the plurality of anchors may be sterile.

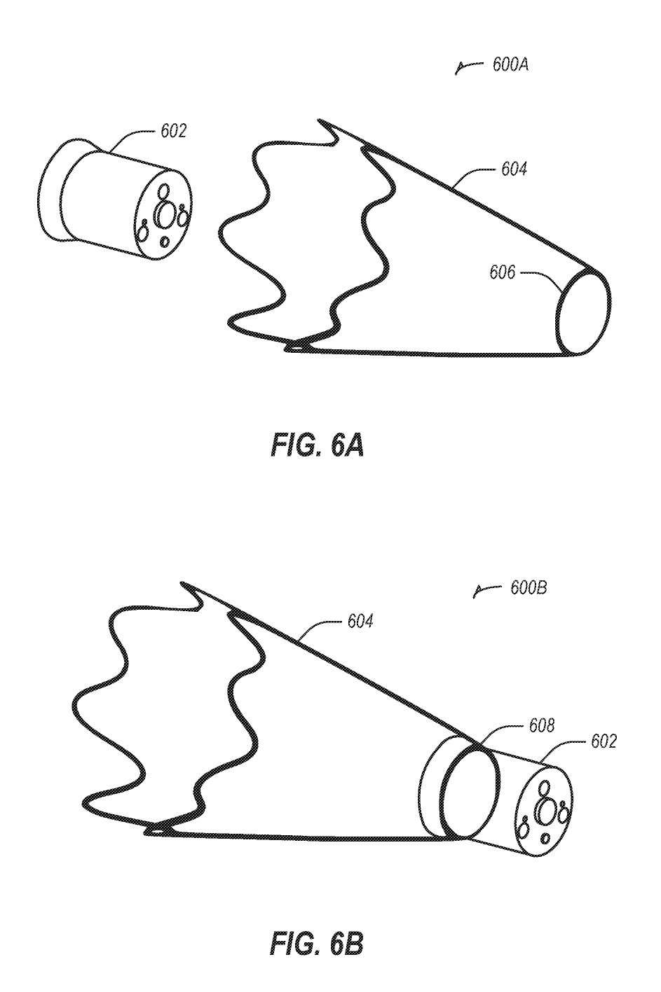

FIGS. 6A-6B illustrate sterile draping components in a first configuration 600A and a second configuration 600B in accordance with some embodiments. The sterile draping components include a sterile end effector 602 and a sterile robotic drape 604. FIG. 6A illustrates the components separated from each other not attached) in the first configuration 600A. The sterile end effector 602 may be inserted into the sterile robotic drape 604 through aperture 606. Once inserted and in the second configuration 600B, the sterile robotic drape 604 may be affixed to the sterile end effector 602, such as using tape 608 to secure the sterile robotic drape 604 to the sterile end effector 602 (e.g., at the aperture 606).

In an example, the sterile robotic drape 604 may be disposable. For example, the sterile end effector 602 may be inserted into the sterile robotic drape 604 for a surgery, and then the sterile robotic drape 604 may be discarded after the surgery. The sterile end effector 602 may be sterilized for a second procedure on a second patient), and a new sterile drape may be used.

In an example, the sterile robotic drape 604 may be affixed to the inserted sterile end effector 602 before installation of the sterile end effector 602 on a robot, such as a robotic arm. After the sterile robotic drape 604 is affixed to the sterile end effector 602, the sterile end effector 602 may be affixed to a robotic arm. The sterile robotic drape 604 may drape around a portion of the robotic arm, such as a portion in a surgical field to keep the surgical field sterile, even if the robotic arm itself is not sterile.

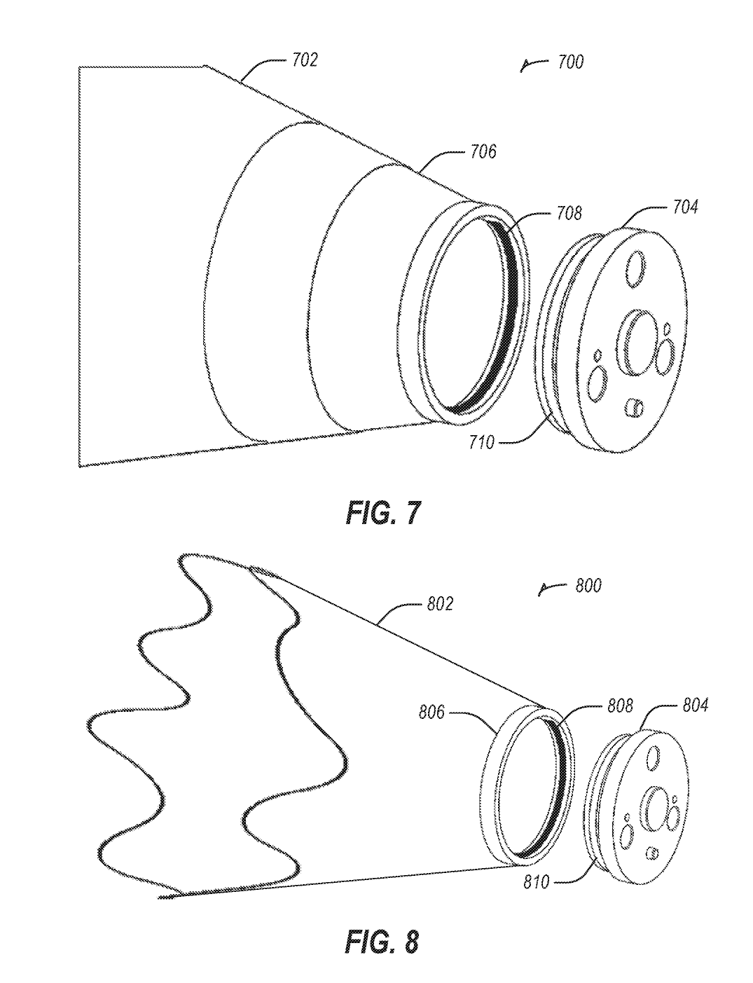

FIG. 7 illustrates a sterile draping system 700 with a cone 706 in accordance with some embodiments. The sterile draping system 700 includes a sterile robotic drape 702, the cone 706, and a sterile end effector 704. The cone 706 may include a gasket 708 at an aperture of the cone 706, the gasket 708 configured to couple with the sterile end effector 704, such as using a gasket inserter 710. The sterile robotic drape 702 may be configured to drape or wrap around a robotic arm, such as a non-sterile robotic arm. The sterile robotic drape 702 may keep the non-sterile robotic arm from being exposed to a sterile field, thereby keeping the sterile field sterile.

In an example, the gasket 708 may be rubber, such as to create a sterile seal around the gasket inserter 710. The sterile seal may prevent non-sterile components inside the sterile robotic drape 702 from entering a sterile field. The sterile end effector 704 may couple with a non-sterile component on a side including the gasket inserter 710 while retaining sterility on a side opposite the gasket inserter 710.

In an example, the cone 706 may be disposable, such as a one-time use cone 706 or cone 706 and sterile robotic drape 702 system. When the cone 706 or cone 706 and sterile robotic drape 702 system is disposable, it may be discarded after use in a sterile field. In an example, the cone 706 may include a plastic cap that may fit around a robotic arm. The cone 706 may be configured such that the sterile robotic drape 702 may be fitted around the cone 706 and pulled tight around the cone 706. For example, the cone 706 may be tapered toward an end that includes the gasket 708, and the sterile robotic drape 702 may include an aperture larger than the end that includes the gasket 708, but smaller than an end opposite the gasket 708, such that the sterile robotic drape 702 may fit snugly around the cone 706 at a tapered location between the end including the gasket 708 and the end opposite the gasket 708. In another example, the cone 706 may be manufactured with the sterile robotic drape 702. For example, the cone 706 and the sterile robotic drape 702 may be glued together, manufactured as a single article, affixed with a heating technique, etc. In yet another example, the cone 706 may be affixed to the sterile robotic drape 702, such as with tape.

The sterile end effector 704 may include fixation screw apertures configured to receive screws. The screws may affix the sterile end effector 704 to a robotic arm. The sterile end effector 704 may include an alignment aperture configured to receive an alignment peg and align the sterile end effector 704 with a robotic arm. The sterile end effector 704 may include an alignment peg configured to align the sterile end effector 704 with the robotic arm. In an example, the sterile end effector 704 may include two or more alignment apertures, two or more alignment pegs, or one or more alignment apertures and one or more alignment pegs.

Affixing the sterile end effector 704 to the sterile robotic drape 702 using the gasket 708 of the cone 706 and the gasket inserter 710 of the sterile end effector 704 may be completed before installation of the sterile draping system 700 on a robot. The sterile draping system 700 may then be affixed to a robot, such as a robotic arm (e.g., using sterile screws, etc). In an example, gaps may be sealed (e.g., gaps surrounding sterile screws or in apertures).

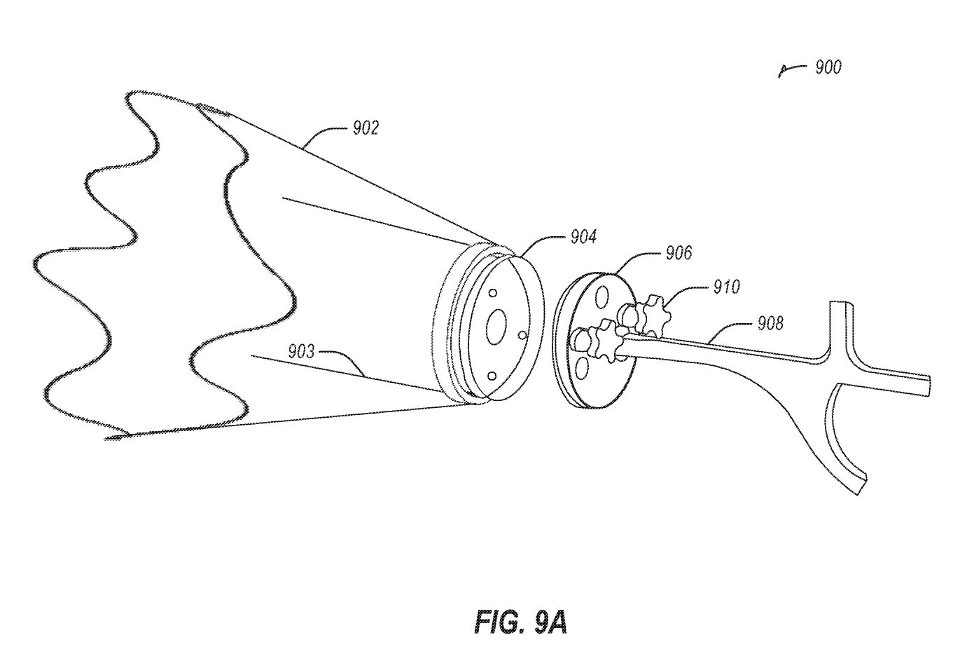

FIG. 8 illustrates a sterile draping system 800 with a ring 806 in accordance with some embodiments. The sterile draping system 800 includes a drape 802, a ring 806, and a sterile end effector 804. The ring 806 may include a gasket 808. The gasket 808 may be configured to couple with the sterile end effector 804, such as using a gasket inserter 810. The drape 802 may be configured to drape or wrap around a robotic arm, such as a non-sterile robotic arm. The drape 802 may keep the non-sterile robotic arm from being exposed to a sterile field, thereby keeping the sterile field sterile.

In an example, the gasket 808 may be rubber, such as to create a sterile seal around the gasket inserter 810. The sterile seal may prevent non-sterile components inside the drape 802 from entering a sterile field. The sterile end effector 804 may couple with a non-sterile component on a side including the gasket inserter 810 while retaining sterility on a side opposite the gasket inserter 810.

The ring 806 and the drape 802 may be manufactured together. For example, the ring 806 and the drape 802 may be glued together, manufactured as a single article, affixed with a heating technique, etc. In yet another example, the ring 806 may be affixed to the drape 802, such as with tape. The ring 806 or the drape 802 may be disposable. The ring 806 or the drape 802 may be made of plastic or the gasket 808 may be made of rubber. The ring 806 or the drape 802 may be disposable.

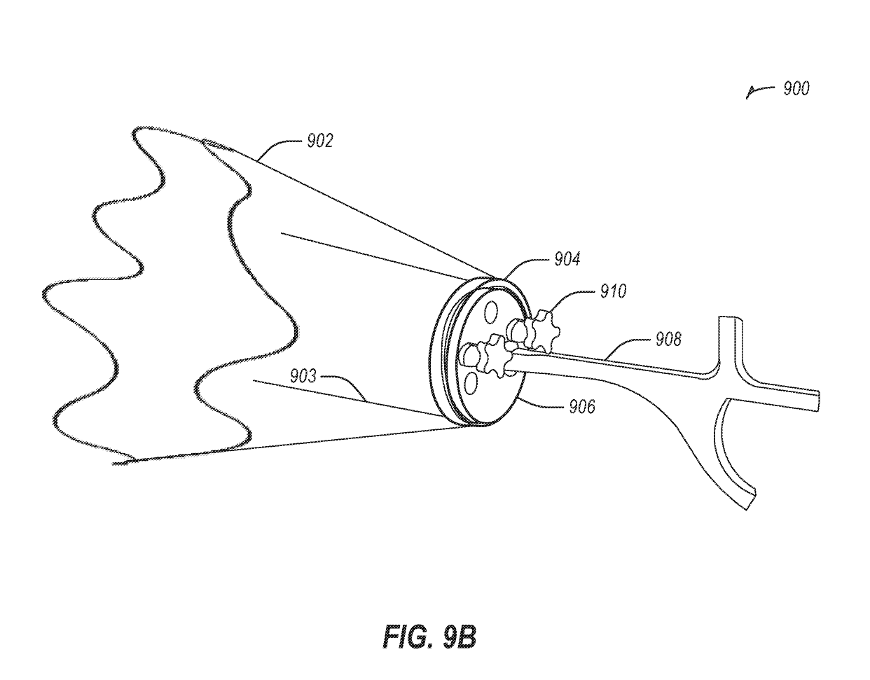

FIGS. 9A-9B illustrate a sterile drape and plate system 900 in accordance with some embodiments. The sterile drape and plate system 900 includes a sterile drape 902, a robotic arm 903 (e.g., non-sterile) a sterile plate 904, and a sterile instrument 908. The sterile instrument includes a plate interface 906, and may include an attachment component 910. FIG. 9A shows a configuration with the sterile drape 902, robotic arm 903, and sterile plate 904 attached, with the sterile instrument 908 unattached. In an example, the sterile drape 902 and the sterile plate 904 may be coupled and detachable from the robotic arm 903. FIG. 9B shows a configuration with the sterile instrument 908 attached to the sterile plate 904 (which may include the sterile plate 904 or the sterile instrument 908 being attached to the robotic arm 903). In an example, the sterile instrument 908 is attached to the sterile plate 904 using the attachment component 910 to secure the sterile instrument 908 to the sterile plate 904. The plate interface 906 may rest against or within the sterile plate 904. In an example, the sterile plate 904 is attached to the robotic arm 903 such that the sterile drape 902 is free to drape around the robotic arm 903. The sterile instrument 908 may be inserted, removed, reinserted, changed, etc., without affecting a sterile boundary around the robotic arm 903 (e.g., a second sterile instrument may in attached to the sterile plate 904 without causing the sterile boundary to be breached).

In an example, the sterile plate 904 may be an embedded robot interface to couple with the robotic arm 903. The sterile plate 904 may include a sterile plate face (e.g., the face that couples with the plate interface 906). The sterile plate 904 may include a non-sterile face (e.g., the face that couples with the robotic arm 903). The embedded robot interface may interface with the sterile instrument 908. In an example, the sterile instrument 908 attaches to the sterile plate 904 using the plate interface 906 to couple with the sterile plate face.

Example 1 is a system comprising: a robotic arm including an end effector receiver with a plurality of apertures; a sterile robotic drape having a first sterile side and a second non-sterile side opposite the first sterile side, the sterile robotic drape including an embedded robot interface; and a sterile robotic end effector configured to couple to the end effector receiver of the robotic arm via the embedded robot interface.

In Example 2, the subject matter of Example 1 includes, wherein the robotic arm is non-sterile and is configured to connect to the embedded robot interface on the second non-sterile side.

In Example 3, the subject matter of Examples 1-2 includes, wherein the embedded robot interface includes a plurality of anchors on the second non-sterile side of the sterile robotic drape.

In Example 4, the subject matter of Example 3 includes, wherein the plurality of anchors are configured to couple with the plurality of apertures.

In Example 5, the subject matter of Example 4 includes, wherein the plurality of anchors are configured to receive a plurality of screws to couple the sterile robotic end effector to the embedded robot interface.

In Example 6, the subject matter of Example 5 includes, wherein the plurality of screws cause the plurality of anchors to expand within the plurality of apertures.

In Example 7, the subject matter of Examples 4-6 includes, wherein, when the plurality of anchors are coupled to the plurality of apertures, rotation of the embedded robot interface with respect to the robotic arm is prevented.

In Example 8, the subject matter of Examples 1-7 includes, wherein the plurality of apertures include a first alignment aperture, the embedded robot interface includes a second alignment aperture, and the sterile robotic end effector includes a third alignment aperture, wherein the first, second, and third alignment apertures are configured to align to receive an alignment peg.

In Example 9, the subject matter of Examples 1-8 includes, wherein an alignment peg is integrated into the sterile robotic end effector, the alignment peg configured to be received by the robotic arm via the embedded robot interface.

In Example 10, the subject matter of Examples 1-9 includes, wherein the sterile robotic end effector is configured to couple to the embedded robot interface on the first sterile side of the sterile robotic drape.

In Example 11, the subject matter of Examples 1-10 includes, wherein the embedded robot interface is a plate including a sterile plate face configured to interface with a sterile instrument, the sterile instrument attaching to the plate using a plate interface to couple with the sterile plate face.

Example 12 is a system comprising: a sterile robotic drape having a first sterile side and a second non-sterile side opposite the first sterile side; a robot interface embedded in the sterile robotic drape, the robot interface including a first face on the first sterile side and a second face on the second non-sterile side; and a plurality of anchors on the second face of the robot interface, the plurality of anchors configured to: couple with a plurality of apertures in a robotic arm; and receive a plurality of screws to couple a sterile robotic end effector to the robotic arm via the robot interface.

In Example 13, the subject matter of Example 12 includes, wherein the plurality of screws cause the plurality of anchors to expand within the plurality of apertures.

In Example 14, the subject matter of Examples 12-13 includes, wherein, when the plurality of anchors are coupled to the plurality of apertures, rotation of the embedded robot interface with respect to the robotic arm is prevented.

In Example 15, the subject matter of Examples 12-14 includes, wherein the robotic arm is non-sterile and is configured to connect to the embedded robot interface on the second non-sterile side.

In Example 16, the subject matter of Examples 12-15 includes, wherein the plurality of screws cause the plurality of anchors to expand within the plurality of apertures.

In Example 17, the subject matter of Examples 12-16 includes, wherein when the plurality of anchors are coupled to the plurality of apertures, rotation of the embedded robot interface with respect to the robotic arm is prevented.

Example 18 is a method of preparing a robot for use within a sterile surgical environment, the method comprising: aligning an embedded robot interface portion of a sterile robot drape with a first end of a robotic arm; coupling a sterile end effector to the first end of the robotic arm through the embedded robot interface portion of the sterile robot drape thereby securing the sterile robot drape to the robotic arm; and draping a portion of the robotic arm extending from the first end of the robotic arm with the sterile robot drape.

In Example 19, the subject matter of Example 18 includes, wherein coupling the sterile end effector to the first end of the robotic arm through the embedded robot interface portion of the sterile robot drape includes coupling the robotic arm to a non-sterile side of the embedded robot interface portion and coupling the sterile end effector to a sterile side of the embedded robot interface portion.

In Example 20, the subject matter of Examples 18-19 includes, wherein coupling the sterile end effector to the first end of the robotic arm through the embedded robot interface portion of the sterile robot drape includes securing a plurality of anchors on the embedded robot interface portion to a plurality of apertures in the first end of the robotic arm using a plurality of screws.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A system comprising:
a robotic arm including an end effector receiver with a plurality of apertures;
a sterile robotic drape having a first sterile side and a second non-sterile side opposite the first sterile side, the sterile robotic drape including an embedded robot interface; and
a sterile robotic end effector configured to couple to the end effector receiver of the robotic arm via the embedded robot interface.

2. The system of claim 1, wherein the robotic arm is non-sterile and is configured to connect to the embedded robot interface on the second non-sterile side.

3. The system of claim 1, wherein the embedded robot interface includes a plurality of anchors on the second non-sterile side of the sterile robotic drape.

4. The system of claim 3, wherein the plurality of anchors are configured to couple with the plurality of apertures.

5. The system of claim 4, wherein the plurality of anchors are configured to receive a plurality of screws to couple the sterile robotic end effector to the embedded robot interface.

6. The system of claim 5, wherein the plurality of screws cause the plurality of anchors to expand within the plurality of apertures.

7. The system of claim 4, wherein, when the plurality of anchors are coupled to the plurality of apertures, rotation of the embedded robot interface with respect to the robotic arm is prevented.

8. The system of claim 1, wherein the plurality of apertures include a first alignment aperture, the embedded robot interface includes a second alignment aperture, and the sterile robotic end effector includes a third alignment aperture, wherein the first, second, and third alignment apertures are configured to align to receive an alignment peg.

9. The system of claim 1, wherein an alignment peg is integrated into the sterile robotic end effector, the alignment peg configured to be received by the robotic arm via the embedded robot interface.

10. The system of claim 1, wherein the sterile robotic end effector is configured to couple to the embedded robot interface on the first sterile side of the sterile robotic drape.

11. The system of claim 1, wherein the embedded robot interface is a plate including a sterile plate face configured to interface with a sterile instrument, the sterile instrument attaching to the plate using a plate interface to couple with the sterile plate face.

12. A system comprising:
a sterile robotic drape having a first sterile side and a second non-sterile side opposite the first sterile side;
a robot interface embedded in the sterile robotic drape, the robot interface including a first face on the first sterile side and a second face on the second non-sterile side; and
a plurality of anchors on the second face of the robot interface, the plurality of anchors configured to:
couple with a plurality of apertures in a robotic arm; and
receive a plurality of screws to couple a sterile robotic end effector to the robotic arm via the robot interface.

13. The system of claim 12, wherein the plurality of screws cause the plurality of anchors to expand within the plurality of apertures.

14. The system of claim 12, wherein, when the plurality of anchors are coupled to the plurality of apertures, rotation of the embedded robot interface with respect to the robotic arm is prevented.

15. The system of claim 12, wherein the robotic arm is non-sterile and is configured to connect to the embedded robot interface on the second non-sterile side.

16. The system of claim 12, wherein the plurality of screws cause the plurality of anchors to expand within the plurality of apertures.

17. The system of claim 12, wherein when the plurality of anchors are coupled to the plurality of apertures, rotation of the embedded robot interface with respect to the robotic arm is prevented.

18. A method of preparing a robot for use within a sterile surgical environment, the method comprising:
aligning an embedded robot interface portion of a sterile robot drape with a first end of a robotic arm;
coupling a sterile end effector to the first end of the robotic arm through the embedded robot interface portion of the sterile robot drape thereby securing the sterile robot drape to the robotic arm; and
draping a portion of the robotic arm extending from the first end of the robotic arm with the sterile robot drape.

19. The method of claim 18, wherein coupling the sterile end effector to the first end of the robotic arm through the embedded robot interface portion of the sterile robot drape includes coupling the robotic arm to a non-sterile side of the embedded robot interface portion and coupling the sterile end effector to a sterile side of the embedded robot interface portion.

20. The method of claim 18, wherein coupling the sterile end effector to the first end of the robotic arm through the embedded robot interface portion of the sterile robot drape includes securing a plurality of anchors on the embedded robot interface portion to a plurality of apertures in the first end of the robotic arm using a plurality of screws.
